(19) United States
(12) Patent Application Publication
(10) Pub. No.: US 2023/0029474 A1
(43) Pub. Date: Feb. 2, 2023

(54) MACHINE VISION FOR CHARACTERIZATION BASED ON ANALYTICAL DATA

(71) Applicant: Dow Global Technologies LLC, Midland, MI (US)

(72) Inventors: James Wade, Midland, MI (US); Alix Schmidt, Midland, MI (US)

(73) Assignee: Dow Global Technologies LLC, Midland, MI (US)

(21) Appl. No.: 17/786,180

(22) PCT Filed: Dec. 1, 2020
(86) PCT No.: PCT/US2020/062683

§ 371 Date: Jun. 16, 2022

Related U.S. Application Data
(60) Provisional application No. 62/949,909, filed on Dec. 18, 2019.

(51) Int. Cl. G16C 20/70; G16C 20/30; G16C 20/10

(52) U.S. Cl. CPC G16C 20/70; G16C 20/30; G16C 20/10

(57) ABSTRACT

Machine vision technology can be used to predict a property of a product generated by a chemical process. The prediction can be based on an analytical characterization of the chemical process or the product generated by the chemical process with a detector that generates series data. The series data can be converted to an image and input to an artificial neural network (ANN) trained to predict the property of the product based on the image. A prediction of a property of the product can be received from the ANN and used to adjust the chemical process or to determine whether to reject the product.

### TECHNICAL FIELD

The present disclosure relates to machine vision for characterization based on analytical data. Such techniques can be particularly useful to predict product properties in order to adjust a chemical process used to produce the product or to determine whether to reject the product.

### BACKGROUND

Artificial neural networks (ANNs) are networks that can process information by modeling a network of neurons, such as neurons in a human brain, to process information (e.g., stimuli) that has been sensed in a particular environment. Similar to a human brain, neural networks typically include a multiple neuron topology (e.g., that can be referred to as artificial neurons). An ANN operation refers to an operation that processes, to perform a given task, inputs using artificial neurons. The ANN operation may involve performing various machine learning algorithms to process the inputs. Example tasks that can be processed by performing ANN operations can include machine vision, speech recognition, machine translation, social network filtering, and/or medical diagnosis.

Chromatography, spectroscopy, and many other analytical characterization methods can produce series data, such as time series or paired x-y series data types. Separations can be useful for material characterization. For example, size-exclusion chromatography, such as gel permeation chromatography (GPC), through careful calibration with molecular weight standards or in combination with a molecular weight sensitive detector such as laser light scattering, can provide a quantitative molecular weight distribution of a polymer sample. Molecular weight distribution can predict many physical properties of polymeric materials. Tailoring the molecular weight distribution is beneficial in polymer manufacturing. For example, improvements in GPC data analysis can improve process control or structural elucidation.

### SUMMARY OF THE DISCLOSURE

The present disclosure is directed to using improvements in machine vision technology to predict a property of a product generated by a chemical process. The prediction can be based on an analytical characterization of the chemical process or the product generated by the chemical process with a detector that generates series data. The series data can be converted to an image and input to an artificial neural network (ANN) trained to predict the property of the product based on the image. A prediction of a property of the product can be received from the ANN and used to adjust the chemical process or to determine whether to reject the product.

As a specific example, the effectiveness of machine vision models for applications in process chemometrics and analytical chemistry is described herein. Images of GPC data collected from chemical products can be used for classification problems (e.g., good versus bad chemical product) and/or to predict product properties. The present disclosure provides improved model performance compared to the use of the summary statistics from the GPC data (e.g., number average molecular weight and weight average molecular weight).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1A illustrates one example approach that plots the detector response on a separate panel with the scale adjusted for each detector.

FIG. 1B illustrates an example approach that overlays detector response from the three detectors onto a single plot.

FIG. 1C illustrates data from FIG. 1A after a Gramian angular summation field (GASF) transformation.

FIG. 1D illustrates data from FIG. 1B after a GASF transformation.

FIG. 2 illustrates an example of a GASF transformation of data.

FIG. 3 illustrates a schematic of the network used according to at least one embodiment of the present disclosure.

FIG. 4A illustrates a set of 100 GPC runs over a period of years without alignment to a solvent peak.

FIG. 4B illustrates the set of 100 GPC runs over the period of years with alignment to the solvent peak.

FIG. 5A illustrates a histogram according to some previous approaches for the weight average molecular weight of chemical product batches including some batches known to be bad.

FIG. 5B illustrates a histogram according to some previous approaches for the number average molecular weight of the chemical product batches including some batches known to be bad.

FIG. 6 illustrates principal component analysis clustering for chromatograms indicated by chemical product quality.

FIG. 7 illustrates a schematic of a workflow for machine vision for chromatography.

FIG. 8 illustrates a comparison of predicted versus actual chemical product property weight percentage levels using images of chromatogram overlays trained with a machine learning architecture according to at least one embodiment of the present disclosure.

FIG. 9 illustrates an example of a system for machine vision for characterization based on analytical data.

FIG. 10 illustrates an example machine within which a set of instructions, for causing the machine to perform various methodologies discussed herein, can be executed.

### DETAILED DESCRIPTION

Deep learning is a type of machine learning that has been enabled by improvements in computational power, data availability, and software tools. Deep learning can apply ANNs to accomplish tasks once thought impossible for a computer to perform. The “deep” of deep learning refers to the use of multiple layers in an ANN. These layers extract successively higher order features from a raw input. For machine vision, examples of lower order features of an input image include edges or color. Higher order features learned at deeper layers in the network may be objects like faces or hand-written digits.

Open source, fully trained networks have been built on databases containing many millions of images. These networks can work with new data via transfer learning, meaning although millions of data points were required to build the initial network, adapting a network to a new use requires much less data. According to at least one embodiment of the present disclosure, pretrained deep learning networks, such as a 2-dimenionsal image input network, can be used for the paired x-y data produced by analytical characterization methods. Conversion of a GPC chromatogram to an image can enable classification by an ANN with above 96% prediction accuracy. Conversion of analytical data (such as GPC data) to an image can be done, for example, by arrayed images of x-y paired data into a line plot. Another example is a GASF transformation of the analytical data (e.g., detector response or y-values) into a two-dimensional matrix that is then colored by the value of each matrix entry. As used herein, an image can refer to a visual or optical representation of something (e.g., a visual representation of data). An image can also refer to the data defining the image, (e.g., when the image is stored in a tangible machine-readable medium). For example, an image can refer to a visual representation displayed on a computer screen or to the electronic file that includes the data defining the image displayed on the screen. Conversion of data to an image means that the data is converted from a non-image format to an image format suitable for use with an ANN trained to predict a property of the product based on the image.

Embodiments of the present disclosure are extendable to combinations of chromatographic, spectral, process, and other data with very minimal subject-matter expertise required. This presents a much faster and more readily leveraged method to use all available data. There are a range of data sources that can be applicable to various embodiments of the present disclosure, such as chemical and physical characterization techniques. Although GPC is described with respect to various examples herein, embodiments are not so limited. Other chemical and physical characterization techniques can be used. Examples of such techniques include size-exclusion chromatography (e.g., GPC), liquid chromatography, gas chromatography, thermal gradient chromatography, calorimetry, rheology, optical spectroscopy, mass spectrometry, viscometry, particle sizing, or nuclear magnetic resonance spectroscopy. This list is not exhaustive. Rather, embodiments of the present disclosure can apply to any measurement method matching the analytical and/or series data described herein.

As used herein, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected and, unless stated otherwise, can include a wireless connection.

As will be appreciated, elements shown in the various embodiments herein can be added, exchanged, and/or eliminated so as to provide a number of additional embodiments of the present disclosure. In addition, as will be appreciated, the proportion and the relative scale of the elements provided in the figures are intended to illustrate certain embodiments of the present invention and should not be taken in a limiting sense.

GPC data structure is an array of x-y data. The x-axis is either time (usually minutes) or volume (usually milliliters “mL”). The y-axis is the detector response, which may consist of multiple detectors. The change in detector response as a function of time (or volume) provides the necessary information to determine molecular weight distribution of a given sample. This data structure is a type of time series because the data are ordered by elution time. One example of time series data analysis is time series forecasting, which uses historical data of a set of variables over time to predict the future values of those variables for a set period in the future. Predicting the future values at a later retention volume may not be useful for GPC. At least one embodiment of the present disclosure includes time series classification or time series regression. Time series classification involves the classification of GPC data into a predetermined set of categories, such as for lot quality discrimination (e.g., good versus bad material), which can be used to determine whether to reject a product produced by a chemical process. Time series regression performs the same underlying task, but the prediction output is a continuous variable, such as predicting viscosity or melt index.

A univariate time series X=[x₁, x₂, . . . , x_{T}] is an ordered set of real values. The length of X is equal to the number of real values T. A multivariate time series is defined as, X=[X₁, X₂, . . . , X_{M}] consists of M different univariate time series with XᵢϵR^{T}. A dataset D=(X₁, Y₁), (X₂, Y₂), . . . , (X_{N}, Y_{N}) is a collection of pairs (Xᵢ, Yᵢ) where Xᵢ could either be a univariate or multivariate time series. An example of this type of data is GPC, where X is the retention volume and Y is the detector(s) response.

Traditionally various statistics have been used to summarize these high dimensional data to a manageable size. Conventional approaches for analysis of GPC data rely on summary statistics to describe a molecular weight distribution (e.g., number average molecular weight Mₙ, weight average molecular weight M_{w}, dispersity B, area under a peak for spectroscopic data, or moduli from dynamic mechanical analysis data). However, in some cases these summary statistics do not have the precision and/or accuracy to capture the level of detail in the material that the full data set includes. An example is small, subtle features such as peak shoulders. In these cases, it is advantageous to use all the analytical data available in a multivariate analysis rather than summary statistics. The field of analytical chemometrics has previously used methods such as principal components analysis or partial least squares regression to utilize entire spectra in near-infrared or Fourier transform infrared spectroscopy methods but have not extended this multivariate approach to other analytical tests. Additionally, methods to combine analytical data from multiple methods or with other sources such as process data have not been fully developed. Challenges regarding data balance can hide valuable correlations from smaller variable sets.

At least one embodiment of the present disclosure includes a new approach for analysis of chromatographic data that uses images as inputs in place of summary statistics or digitized time-intensity arrays. Leveraging the success of machine vision applications, an ANN, such as a deep neural network can be trained on images of GPC data for both classification and regression tasks. In comparison to conventional GPC data analysis, this requires significantly more computational resources and larger data sets for successful implementation.

There is a wide array of silicone materials with complex polymeric structures. Chromatography, primarily GPC, can be used to characterize the quality of these materials. Silicone materials can be used as raw materials for producing other products. However, problems with raw material lots that show no unusual properties via GPC summary statistics (e.g., Mₙ, M_{w}) or other lot acceptance requirements (e.g., silanol), have nonetheless caused problems downstream.

The problem of adequately characterizing the composition and performance of advanced materials has been pervasive across many applications in silicones. A quality control gap arises between the product by process approach versus obtaining quantitative property metrics on which lots of material can be specified. Analytical characterization specialists could enable process improvement through a better understanding of the target material and its properties.

GPC data collected for in-process analysis of silicone materials over a period of years is used as an example. The ANN can be used to predict the quality of silicone polymer raw material as judged by a known manufacturing upset and to predict final product properties, namely vinyl and silanol percentages. Previous approaches, including GPC data reduced to summary statistics, have not successfully modelled the classification of polymer or downstream product quality.

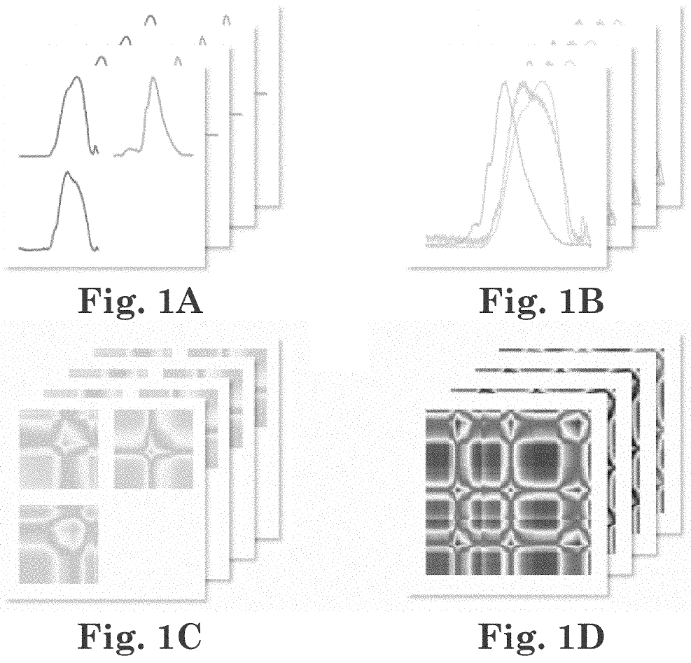

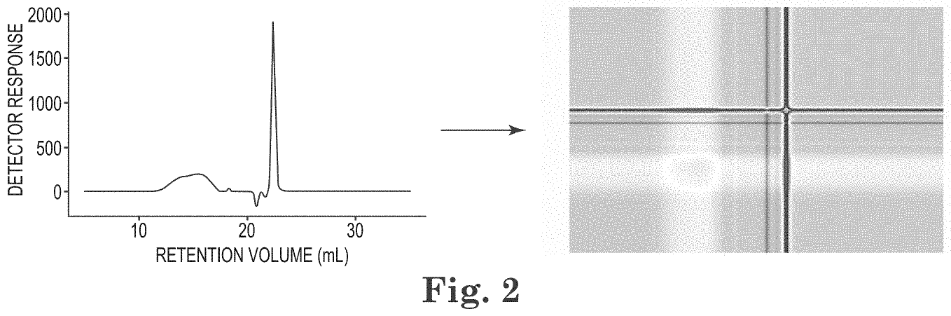

Images can be generated for samples using a variety of approaches. FIG. 2A illustrates one example approach that plots the detector response on a separate panel with the scale adjusted for each detector. This is referred to as the faceted plot. FIG. 1B illustrates an example approach that overlays detector response from the three detectors onto a single plot. FIG. 1C illustrates data from FIG. 1A after a GASF transformation. FIG. 1D illustrates data from FIG. 1B after a GASF transformation. The GASF transformation is an alternative encoding of the data.

FIG. 2 illustrates an example of a GASF transformation of data. The GASF transformation can include three steps of data augmentation. First, given a time series X={x₁, x₂, . . . , xₙ} of n observations, rescale X so that all values fall on the interval [0, 1].

$${\tilde{x}}_{0} = \frac{{x}_{i} - min ( X )}{max ( X ) - min ( X )}$$

Second, transform rescaled time series X in polar coordinates using the value \tilde{x}ᵢ as the angular cosine and time as the radius. In the equation below, N is a constant that regularizes the span of the polar coordinates.

$$\{ \begin{matrix}ϕ = arccos ( {\tilde{x}}_{i} ) , - 1 \leq {\tilde{x}}_{i} \leq 1 \\ r = \frac{{t}_{i}}{N}\end{matrix}$$

Third, take the GASF.

$$\begin{matrix}GASF = [ cos ( {ϕ}_{i} + {ϕ}_{j} ) ] \\ = \cdot \tilde{X} - {\sqrt{I - {\tilde{X}}^{2}}}^{′} \cdot \sqrt{I - {\tilde{X}}^{2}}\end{matrix}$$

In the equation above, l is the unit row vector. Polar coordinates preserve absolute temporal relations whereas Cartesian coordinates do not. With the polar coordinates, the angular cosine is the value (e.g., detector response) and the radius is the time step (e.g., retention volume). One advantage of the polar coordinates is preservation of absolute temporal relations. The Gramian transformation reduces sparsity in the images fed into the network as compared to cartesian time series plots. In this case, sparsity refers to the proportion of whitespace in an image of the chromatogram. For the signal overlay images (FIG. 1B), greater than 93% of pixels are white. The Gramian transformation reduces the number of white pixels to nearly zero. Imputation may be improved by this method compared to raw time series data.

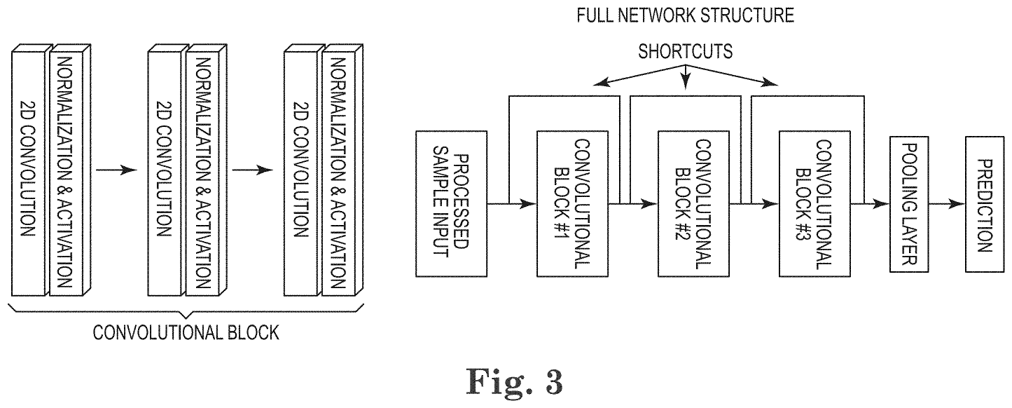

FIG. 3 illustrates a schematic of the network used according to at least one embodiment of the present disclosure. The network has eleven layers overall with three convolutional blocks. Each block contains three convolutional layers, summing to nine convolutional layers in total. However, embodiments are not limited to any particular number of layers or convolutional blocks. After each convolution, there can be batch normalization and activation steps. The batch normalization step can normalize the layer outputs to have a mean close to zero and a standard deviation close to one. The normalization method can be used as an alternative to dropout to limit overfitting. The activation step can use a rectified linear unit as the activation function. The penultimate layer can perform a global average pooling operation. The last layer is the prediction step. The shortcuts shown in the network schematic refer to residual network connection. The residual network bypass convolution blocks and have been shown to significantly improve training time for deep networks by solving to the problem of vanishing gradients during network optimization. For example, these shortcuts allowed for network optimization and error reduction to be passed through many layers of a deep neural network.

Hyperparameters for the model can be adjusted to improve model accuracy for a given embodiment of the present disclosure. As a non-limiting example, the CNN layer filters can be 32, 64, and 64 for the layers in code blocks 1, 2, and 3, respectively. A kernel size of 8×8, 3×3, and 1×1 can be used for layers 1, 2, and 3 within a given convolutional block, respectively. Data analysis can be performed using available tools.

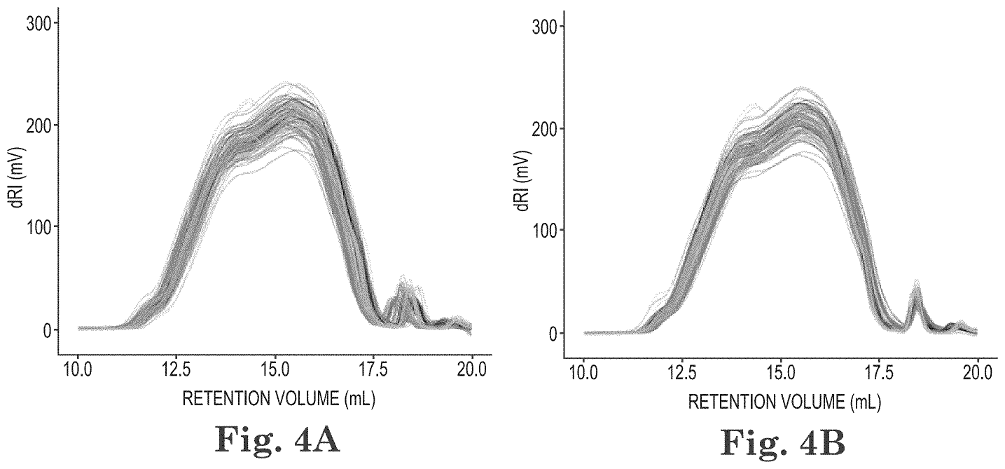

FIG. 4A illustrates a set of 100 GPC runs over a period of years without alignment to a solvent peak. The peak appearing at a retention volume between 17.5 and 18.5 mL corresponds to a known monomer species with consistent size. Because the monomer is structurally similar across all samples, the corresponding peak in the GPC should overlap across all runs analyzed with the same method. The peaks do not align well in FIG. 4A, indicating that the GPC results have some drift over months or years. FIG. 4B illustrates the set of 100 GPC runs over the period of years with alignment to the solvent peak. By aligning to this solvent peak, the overall run alignment as observed by the monomer peak is much improved. The aligned GPC results were used for subsequent analysis. The data presented in FIGS. 4A-4B shows results for aligned chromatograms, however embodiments are not limited to such alignment to generate accurate machine learning models. The accuracy of the classification model may be indistinguishable between aligned and unaligned data in terms of model accuracy.

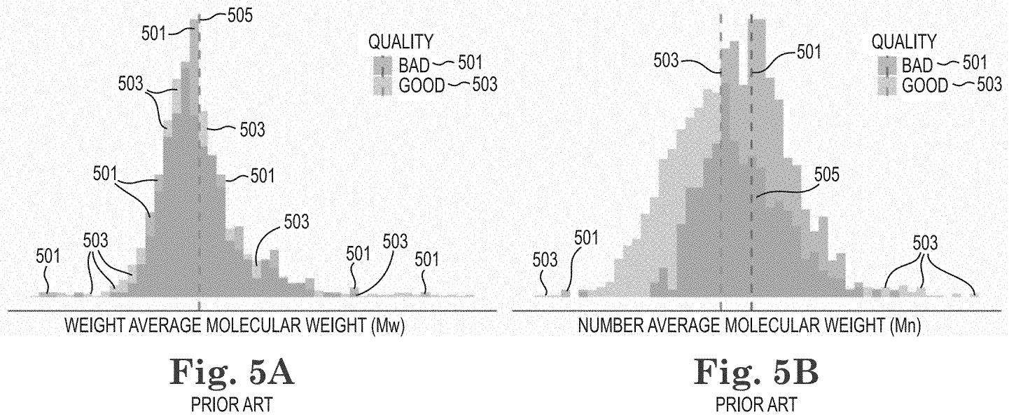

Previous efforts at analysis of summary statistics were not able to identify GPC characteristics that could discriminate good 503 versus bad 501 chemical products in these batches. FIG. 5A illustrates a histogram according to some previous approaches for the weight average molecular weight of chemical product batches including some batches known to be poor performers. The dashed lines in FIGS. 5A-5B represent the mean for that category. The good 503 and bad 501 batches appear to have a nearly identical distribution. The overlap is labeled as 505. In FIG. 5A, the dashed line represents an overlap 505 between the mean for the good 503 and bad 501 chemical product batches. In FIG. 5B, separate dashed lines are illustrated for the mean of the good 503 and bad 501 categories. Control of these properties when producing the chemical product does not mean that the overall product has a similar tightness of the distribution of number average molecular weight values.

FIG. 5B illustrates a histogram according to some previous approaches for the number average molecular weight of the chemical product batches including some batches known to be bad 501. The distribution of number average molecular weights for the chemical product shows a greater difference between good 503 and bad 501 than weight average molecular weight. The bad 501 sample distribution has a higher number average molecular weight on average, meaning that the product distribution is still drifting from batch to batch despite the consistent weight average molecular weight. The number average molecular weight shows more of a difference for good 503 versus bad 501 material, but the significant overlap between the distributions prevents the number average molecular weight from being an accurate discriminator of batch quality.

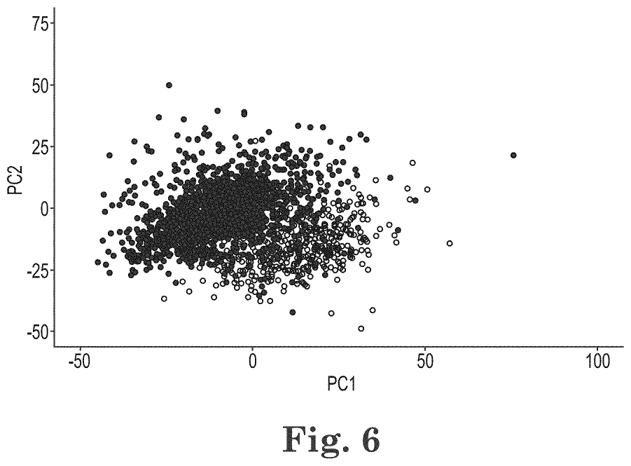

Unsupervised learning can be applied to the data assembled to discern differences in the GPC data across various chemical product lots. An unsupervised learning task is one that models the underlying structure of the data without explicit labels (Y data) for each sample. Such methods can identify previously unknown patterns or features in the data. In this example, the unsupervised learning task can identify patterns in the chromatograms and then use those patterns to separate clusters of samples. These clusters should represent good and bad chemical product lots, but labels are not included in the analysis. One example of a method for unsupervised learning is principal component analysis (PCA), which is a dimension reduction technique that emphasizes data variance. FIG. 6 illustrates principal component analysis clustering for chromatograms indicated by chemical product quality. In FIG. 6, the different chemical product qualities are indicated by filled and open dots due to the limitations of printed patent publications. However, actual chromatograms typically make such distinctions by color. FIG. 6 shows that the PCA-based visualization can show differences in the average good versus bad lot but does not show significant enough separation to discriminate between good and bad for unseen GPC data (to predict the quality of a new lot).

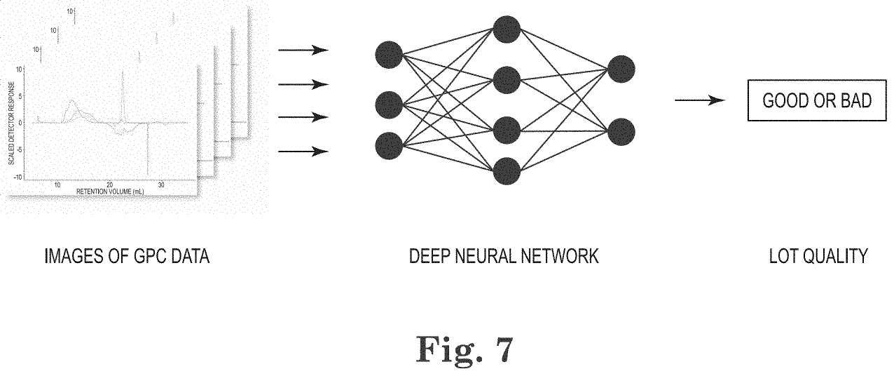

Image classification with ANNs, such as deep neural networks, has been a successful application of machine learning. According to the present disclosure, generated images of the chromatographic data can be used as input images to perform the classification task for the chemical product. FIG. 7 illustrates a schematic of a workflow for machine vision for chromatography. The network architecture for lot classification can train the ANN (“deep neural network”) by vanishing gradients, for example. Network optimization can occur using backpropagation and gradient descent to minimize a defined loss function. Propagating through the ANN can result in a vanishingly small gradient, saturating or degrading model performance. Shortcut connections can be integrated to skip layers in the network allowing gradient propagation therethrough with many layers (e.g., more than 100 layers).

Each layer of the ANN is represented in the simplified image in FIG. 7 as a column of nodes. The nodes, which can correspond to artificial neurons, can receive various inputs. Interconnection regions can couple nodes between different layers as indicated by the lines coupling the nodes in FIG. 7. Nodes can receive inputs from other nodes via the interconnection regions. In at least one embodiment, an interconnection region can couple each node of a first layer with each node of a second layer, however embodiments are not so limited. The ANN can be configured in a training process in which the various connections in the interconnection regions are assigned a weight value or updated with a new weight value that is used for operations or computations at the nodes. The training process may be different depending on a particular application or use of the ANN. For instance, an ANN may be trained for image recognition as described herein, or another processing or computational task.

The ANN can include an output layer, represented by the last column of nodes on the right side of the image. The last column of nodes may be referred to as output nodes. Each of the output nodes can be coupled to receive inputs from the nodes of the previous layer of nodes (to the left). The process of receiving usable outputs at the output layer of output nodes as a result of inputs fed into the nodes at the first layer (the leftmost layer as illustrated in FIG. 7) may be referred to as inference or forward propagation. That is, input signals representative of some real-world phenomena or application may be fed into a trained ANN and through inference that occurs as a result of calculations enabled by various nodes and interconnects, a result may be output. In the case of an ANN trained for image recognition, the input may be signals representative of a chromatogram and the output may be signals representative of the quality of the chemical product indicated by the chromatogram.

A test was performed and the results for classification of the chemical product as either good or bad is summarized according to input image type as follows. In each case, the images were trimmed and normalized. Trimming refers to narrowing the range of retention volumes to only include regions of the chromatogram deemed relevant b subject matter experts. Normalization was performed by scaling the plots to contain values between 0 and 1. When separate GPC curves were input, each quadrant had a single GPC chromatogram. The data preprocessing is not required for the modeling task, but in some instances, it may improve model performance by discarding uninformative data. The test set accuracy was 98.9%. When overlaid GPC curves were input, the GPC curves were overlaid in each image. The test set accuracy was 99.2%. When separate GASF transformations were input, each quadrant had a single GASF transformed GPC signal. The test set accuracy was 99.2%. When single GASF transform images were input, the images were of GASF transforms of linear extension of GPC signals. The test set accuracy was 98.7%. The performance of each image category was excellent, with an accuracy of 99.0±0.2%. Comparing across image input type, there does not appear to be a significant performance difference for any of the inputs. Even the state-of-the-art analytical characterization methods applied to this classification challenge have not revealed an obvious standalone method to assess chemical product.

The network architecture and hyperparameters for regression can be the same as the classification model. To convert from a classification model to regression, the last layer of the neural network can be changed from sigmoid activation with a single node to a layer with no activation and two output nodes. For example, with respect to FIG. 7, the output would be product properties rather than lot quality. Specifically, with respect to a chemical process that produces silicone materials, one of the nodes corresponds to vinyl content, and the other corresponds to silanol content. Multiple output nodes can either predict their variables simultaneously or a different model can be used for each prediction (output node).

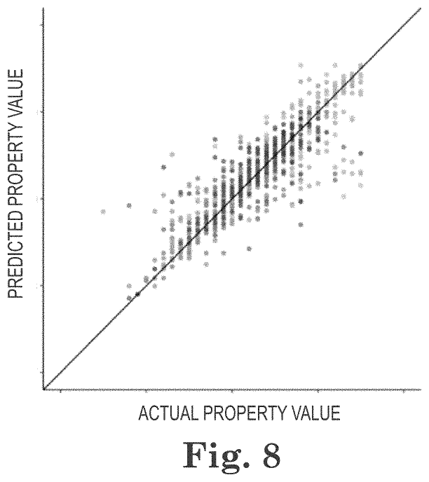

FIG. 8 illustrates a comparison of predicted versus actual chemical product property values using images of chromatogram overlays trained with a machine learning architecture according to at least one embodiment of the present disclosure. The prediction plot demonstrates that quantitative predictions from GPC image data alone can be used to predict chemical product property values with a relative error of 0.7%. This demonstrates a practical application of the machine learning method for a chemical product produced by a chemical process.

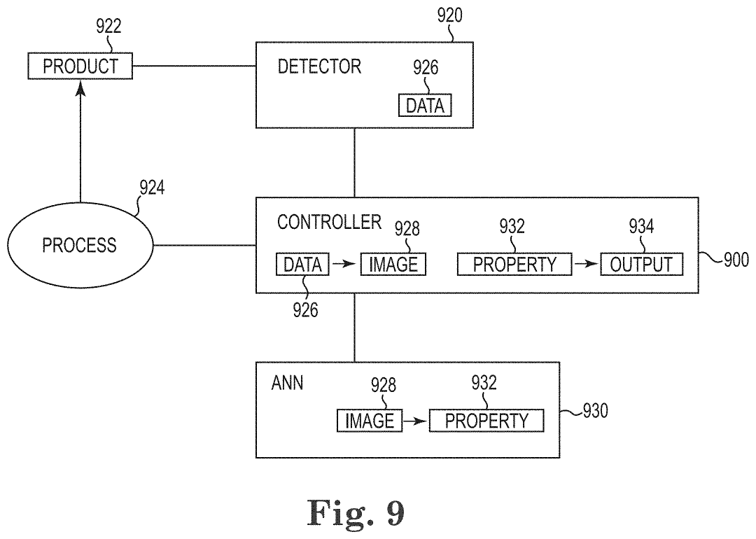

FIG. 9 illustrates an example of a system for machine vision for characterization based on analytical data. The system can include a detector 920 configured to analytically characterize a product 922 generated by a chemical process 924. Examples of the detector 920 include a concentration sensitive detector, a molecular weight sensitive detector, a composition sensitive detector, or combinations thereof. Examples of concentration sensitive detectors include UV absorption, differential refractometer or refractive index detectors, infrared absorption, and density detectors. Examples of molecular weight sensitive detectors include low angle light scattering detectors and multiangle light scattering detectors. Examples of the analytical characterization methods include size-exclusion chromatography, liquid chromatography, gas chromatography, thermal gradient chromatography, calorimetry, rheology, optical spectroscopy, mass spectroscopy, viscometry, particle sizing, and nuclear magnetic resonance spectroscopy. The detector 920 can be configured to generate series data 926 from the analytical characterization. For example, the series data 926 can be multivariate data. The series data 926 can be multivariate, for example, in embodiments including instruments with multiple detectors (e.g., GPC with refractive index and light scattering) or embodiments including multiple instruments, each with at least one detector (e.g., for multiple separate characterizations of the same product). The product 922 can be a polymeric material generated by a chemical process 924.

The system can include an ANN 930 trained with a plurality of images of converted series data from prior products generated by the chemical process 924 to predict a property 932 of the product 922 based on an image 928 converted from the series data 926. The ANN 930 can be pretrained to identify a feature in an image and further trained via transfer learning with a plurality of images of converted series data from prior products generated by the chemical process 924 such that the feature that the ANN 930 is now trained to identify is the property 932 of the product 922. Examples of the property 932 of the product 922 include molecular weight, density, quality, performance, and identification. In at least one embodiment, the ANN 930 can be a two-dimensional image input network. Although illustrated as being separate from the controller 900, in at least one embodiment, the ANN 930 can be implemented by the controller 900. The ANN 930 is described in more detail above.

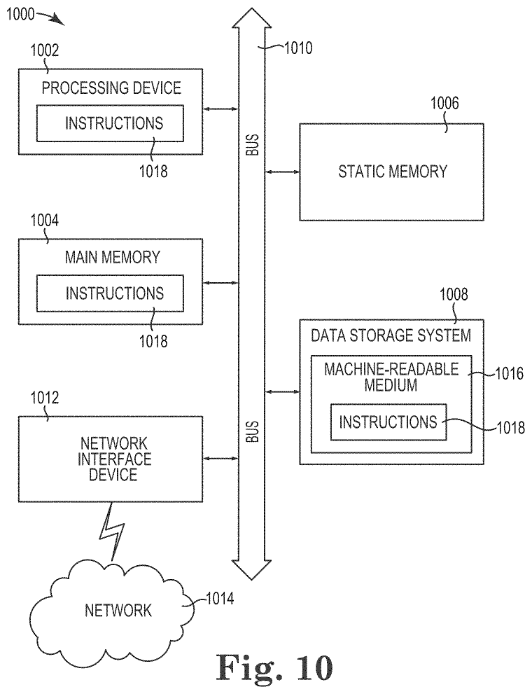

The system can include a controller 900 coupled to the detector 920 and to the ANN 930. Although not specifically illustrated, the controller 900 can include a processor and memory resources storing instructions executable by the controller 900 to perform the functions described herein. An example of the controller 900 is described in more detail with respect to FIG. 10. The controller 900 can be configured to convert the series data 926 to the image 928 and input the image 928 to the ANN 930. In at least one embodiment, the controller 920 can be configured to convert the series data 926 to the image 928 without preprocessing the series data 926. As described herein, conversion of the time series data 926 (such as GPC data) to the image 928 can be done, for example, by arrayed images of x-y paired data into a two-dimensional line plot or a GASF transformation, among other conversion methods. The controller 900 can be configured to receive the prediction of the property 932 of the product 922 from the ANN 930.

The controller 900 can be configured to provide an output 934 based on the prediction of the property 932 (e.g., if the property 932 does not meet a predefined specification for the property 932). An example of the output 934 is an adjustment to the chemical process 924. As such, in at least one embodiment, the controller 900 can be configured to control, or be coupled to other control circuitry that controls the chemical process 924. In such an example, the controller 900 can cause one or more parameters of the chemical process to be adjusted such that the property of chemical products subsequently produced by the chemical process 924 is more likely to be within the predefined specification. As another example, the output 934 from the controller 900 can be used to adjust the chemical process 924 by human intervention (e.g., where a human adjusts the one or more parameters of the chemical process 924 such that the property of chemical products subsequently produced by the chemical process 924 is more likely to be within the predefined specification). The output 934 can be control signals for the chemical process 924, data indicating the acceptability of the product 922, or an indication such as a light or sound indicating the acceptability of the product 922. As another example, the output 934 can be a rejection of the product 922. For example, the controller 900 can provide an indication to an operator that the product 922 should be rejected or the controller 900 can automatically flag the product 922 for rejection. In at least one embodiment, the controller 900 can be configured to both adjust the chemical process 924 and reject the product 922 based on the property 932 of the product.

FIG. 10 illustrates an example machine 1000 within which a set of instructions, for causing the machine 1000 to perform various methodologies discussed herein, can be executed. In various embodiments, the machine 1000 can be analogous to the controller 900 described with respect to FIG. 9. In alternative embodiments, the machine 1000 can be connected (e.g., networked) to other machines in a LAN, an intranet, an extranet, and/or the Internet. The machine 1000 can operate in the capacity of a server or a client machine in client-server network environment, as a peer machine in a peer-to-peer (or distributed) network environment, or as a server or a client machine in a cloud computing infrastructure or environment.

The machine 1000 can be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine 1000 is illustrated, the term “machine” shall also be taken to include any collection of machines that individually or jointly execute a set (or multiple sets) of instructions to perform any one or more of the methodologies discussed herein.

The example machine 1000 includes a processing device 1002, a main memory 1004 (e.g., read-only memory (ROM), flash memory, dynamic random access memory (DRAM) such as synchronous DRAM (SDRAM) or Rambus DRAM (RDRAM), etc.), a static memory 1006 (e.g., flash memory, static random access memory (SRAM), etc.), and a data storage system 1008, which communicate with each other via a bus 1010.

The processing device 1002 represents one or more general-purpose processing devices such as a microprocessor, a central processing unit (CPU), or the like. More particularly, the processing device can be a complex instruction set computing (CISC) microprocessor, reduced instruction set computing (RISC) microprocessor, very long instruction word (VLIW) microprocessor, or a processor implementing other instruction sets, or processors implementing a combination of instruction sets. The processing device 1002 can also be one or more special-purpose processing devices such as an application specific integrated circuit (ASIC), a field programmable gate array (FPGA), a digital signal processor (DSP), network processor, or the like. The processing device 1002 is configured to execute instructions 1018 for performing the operations and steps discussed herein. The machine 1000 can further include a network interface device 1012 to communicate over the network 1014.

The data storage system 1008 can include a machine-readable storage medium 1016 (also known as a computer-readable medium) on which is stored one or more sets of instructions 1018 or software embodying any one or more of the methodologies or functions described herein. The instructions 1018 can also reside, completely or at least partially, within the main memory 1004 and/or within the processing device 1002 during execution thereof by the machine 1000, the main memory 1004 and the processing device 1002 also constituting machine-readable storage media.

In one embodiment, the instructions 1018 include instructions to implement functionality corresponding to the ANN described herein. While the machine-readable storage medium 1016 is shown in an example embodiment to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media, and magnetic media.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Various advantages of the present disclosure have been described herein, but embodiments may provide some, all, or none of such advantages, or may provide other advantages.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

1. A method, comprising:
analytically characterizing a chemical process or a product generated by the chemical process with a detector thereby generating series data;
converting the series data to an image;
inputting the image to an artificial neural network (ANN) trained to predict a property of the product based on the image;
receiving the prediction of the property of the product from the ANN; and
adjusting the chemical process or rejecting the product based on the prediction of the property of the product.

2. The method of claim 1, further comprising adjusting the chemical process and rejecting the product based on the prediction of the property of the product.

3. The method of claim 1, wherein the ANN is pretrained to identify a feature in any image; and
wherein the method further comprises training the ANN via transfer learning with a plurality of images of converted series data from prior products generated by the chemical process such that the feature comprises the property of the product.

4. The method of claim 1, wherein receiving the prediction of the property of the product comprises receiving the prediction of one of a group of properties including molecular weight, density, quality, performance, and identification.

5. The method of claim 1 wherein analytically characterizing the product comprises one of a group of analytical characterizations including, liquid chromatography, gas chromatography, thermal gradient chromatography, size-exclusion chromatography, calorimetry, rheology, optical spectroscopy, mass spectroscopy, viscometry, particle sizing, and nuclear magnetic resonance spectroscopy.

6. The method of claim 1, wherein converting the series data to the image comprises converting the series data to a two-dimensional line plot.

7. The method of claim 1, wherein converting the series data to the image comprises converting the series data to a Gramian angular summation field.

8. The method of claim 1, wherein converting the series data to the image comprises converting the series data without preprocessing the series data.

9. The method of claim 1, wherein inputting the image to the ANN comprises inputting the image to a two-dimensional image input network.

10. A system, comprising:
a detector configured to:
analytically characterize a product generated by a chemical process; and
generate series data from the analytical characterization;
an artificial neural network (ANN) trained with a plurality of images of converted series data from prior products generated by the chemical process to predict a property of the product based on an image converted from the series data; and
a controller coupled to the detector and to the ANN, wherein the controller is configured to:
convert the series data to the image;
input the image to the ANN;
receive the prediction of the property of the product from the ANN; and
adjust the chemical process or reject the product.

11. The system of claim 10, wherein the system includes a plurality of detectors and wherein the series data comprises multivariate data corresponding to the plurality of detectors.

12. The system of claim 10, wherein the detector comprises one of a group of detectors including a concentration sensitive detector, a molecular weight sensitive detector, a composition sensitive detector, and combinations thereof.

13. The system of claim 10, wherein the controller is configured to adjust the chemical process and reject the product.

14. The system of claim 10, wherein the controller is configured to convert the series data to the image without preprocessing the series data.

15. The system of claim 10, wherein the ANN is a two-dimensional image input network.
